(19) United States
(12) Patent Application Publication
(10) Pub. No.: US 2026/0090809 A1
(43) Pub. Date: Apr. 2, 2026

(54) OCCLUSIVE IMPLANT AND METHODS OF MAKING AN OCCLUSIVE FABRIC HAVING SHAPE MEMORY CHARACTERISTICS

(71) Applicant: BOSTON SCIENTIFIC SCIMED, INC., MAPLE GROVE, MN (US)

(72) Inventors: Patrick Willoughby, Shoreview, MN (US); Scott Andrew McCullough, Plymouth, MN (US)

(73) Assignee: BOSTON SCIENTIFIC SCIMED, INC., MAPLE GROVE, MN (US)

(21) Appl. No.: 19/341,220

(22) Filed: Sep. 26, 2025

Related U.S. Application Data
(60) Provisional application No. 63/700,026, filed on Sep. 27, 2024.

(51) Int. Cl. A61B 17/12; A61B 17/00

(52) U.S. Cl. CPC A61B 17/12177; A61B 17/12122; A61B 17/12172; A61B 2017/00526; A61B 2017/00867

(57) ABSTRACT

An occlusive implant may include a framework configured to shift between collapsed and expanded configurations, and an occlusive fabric secured to the framework. The fabric includes shape memory characteristics such that the fabric is configured to shift from a first to a second configuration when exposed to a stimulus. A method of making the fabric includes intertwining a plurality of filaments into the fabric, defining a permanent shape of the fabric in the second configuration, and heat setting the fabric in the first configuration. A method of making a shape memory yarn includes forming a core filament, and wrapping at least one shape memory filament helically around the core filament. The at least one shape memory filament is formed from a shape memory polymer and is configured to shift from a first to a second configuration when exposed to a stimulus.

### CROSS-REFERENCE TO RELATED APPLICATIONS

This application claims the benefit of priority of U.S. Provisional Application No. 63/700,026 filed September 27, 2024, the entire disclosure of which is hereby incorporated by reference.

### TECHNICAL FIELD

The disclosure relates generally to medical devices and more particularly to an occlusive implant having an occlusive fabric and methods of making an occlusive fabric having shape memory characteristics.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Over time, exposed surface(s) of an implant spanning the left atrial appendage may become covered with tissue (a process called endothelization), effectively removing the left atrial appendage from the circulatory system and reducing or eliminating the thrombi which may enter the blood stream from the left atrial appendage.

Of the known medical devices and methods, each has certain advantages and disadvantages. There is an ongoing need to provide alternative medical devices as well as alternative methods for manufacturing and using medical devices.

### SUMMARY

In a first example, an occlusive implant may comprise a framework configured to shift between a collapsed configuration and an expanded configuration, and an occlusive fabric secured to the framework. The occlusive fabric may comprise shape memory characteristics such that the occlusive fabric is configured to shift from a first configuration to a second configuration when exposed to a predetermined stimulus.

In addition, or alternatively, to any example disclosed herein, the occlusive fabric is formed from a plurality of filaments comprising a first subset of filaments formed from a non-shape memory material and a second subset of filaments formed from a shape memory material.

In addition, or alternatively, to any example disclosed herein, the second subset of filaments is evenly distributed within the occlusive fabric.

In addition, or alternatively, to any example disclosed herein, the second subset of filaments is unevenly distributed within the occlusive fabric.

In addition, or alternatively, to any example disclosed herein, the second subset of filaments is concentrated along an edge of the occlusive fabric.

In addition, or alternatively, to any example disclosed herein, the occlusive fabric comprises a plurality of pores.

In addition, or alternatively, to any example disclosed herein, each pore of the plurality of pores has a first cross-sectional area in the first configuration and a second cross-sectional area less than the first cross-sectional area in the second configuration.

In addition, or alternatively, to any example disclosed herein, the occlusive fabric is self-biased toward a collapsed configuration in the first configuration and is self-biased toward an extended configuration in the second configuration.

In addition, or alternatively, to any example disclosed herein, the occlusive fabric is self-biased toward a collapsed configuration in the second configuration.

In addition, or alternatively, to any example disclosed herein, the occlusive fabric comprises a coating formed from a shape memory polymer configured to react to the predetermined stimulus.

In addition, or alternatively, to any example disclosed herein, the predetermined stimulus is a temperature greater than 37 degrees C.

In addition, or alternatively, to any example disclosed herein, the predetermined stimulus is a fluid.

In addition, or alternatively, to any example disclosed herein, and in another example, a method of making an occlusive fabric having shape memory characteristics may comprise: intertwining a plurality of filaments into an occlusive fabric, wherein the occlusive fabric comprises shape memory characteristics such that the occlusive fabric is configured to shift from a first configuration to a second configuration when exposed to a predetermined stimulus; defining a permanent shape of the occlusive fabric in the second configuration; and heat setting the occlusive fabric in the first configuration, wherein the first configuration defines a temporary shape of the occlusive fabric.

In addition, or alternatively, to any example disclosed herein, the plurality of filaments comprises a first subset of filaments formed from a non-shape memory material and a second subset of filaments formed from a shape memory material, wherein the second subset of filaments is configured to react to the predetermined stimulus.

In addition, or alternatively, to any example disclosed herein, the method may comprise coating the occlusive fabric with a shape memory polymer configured to react to the predetermined stimulus.

In addition, or alternatively, to any example disclosed herein, the predetermined stimulus is a fluid or a temperature greater than 37 degrees C.

In addition, or alternatively, to any example disclosed herein, and in another example, a method of making a shape memory yarn may comprise: forming a core filament from a first material comprising a polymer; and wrapping at least one shape memory filament helically around the core filament, wherein the at least one shape memory filament is formed from a second material comprising a shape memory polymer. The at least one shape memory filament may be configured to shift from a first configuration to a second configuration when exposed to a predetermined stimulus.

In addition, or alternatively, to any example disclosed herein, the at least one shape memory filament defines a first outer extent from the core filament in the first configuration and a second outer extent from the core filament greater than the first outer extent in the second configuration.

In addition, or alternatively, to any example disclosed herein, the at least one shape memory filament defines a first spacing between adjacent helical windings in the first configuration and a second spacing between adjacent helical windings less than the first spacing in the second configuration.

In addition, or alternatively, to any example disclosed herein, the predetermined stimulus is a fluid or a temperature greater than 37 degrees C.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-2 illustrate selected aspects of an occlusive implant system comprising an occlusive implant for occluding a left atrial appendage;

FIG. 3 illustrates selected aspects of an occlusive implant comprising occlusive fabric according to the disclosure;

FIG. 4 illustrates selected aspects of occlusive fabric for use with the occlusive implant of FIG. 3;

FIG. 5 illustrates selected aspects of the occlusive implant of FIG. 3 comprising an occlusive fabric according to the disclosure;

FIGS. 6-8 illustrate selected aspects of occlusive fabric according to the disclosure;

FIGS. 9-10 illustrates selected aspects of a shape memory yarn and methods of making the shape memory yarn; and

FIG. 11 illustrates selected aspects of occlusive fabric according to the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

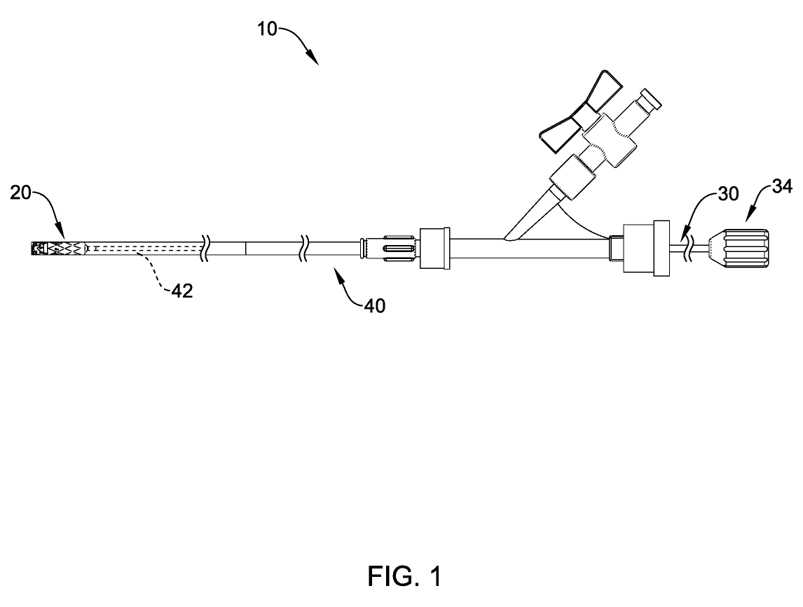

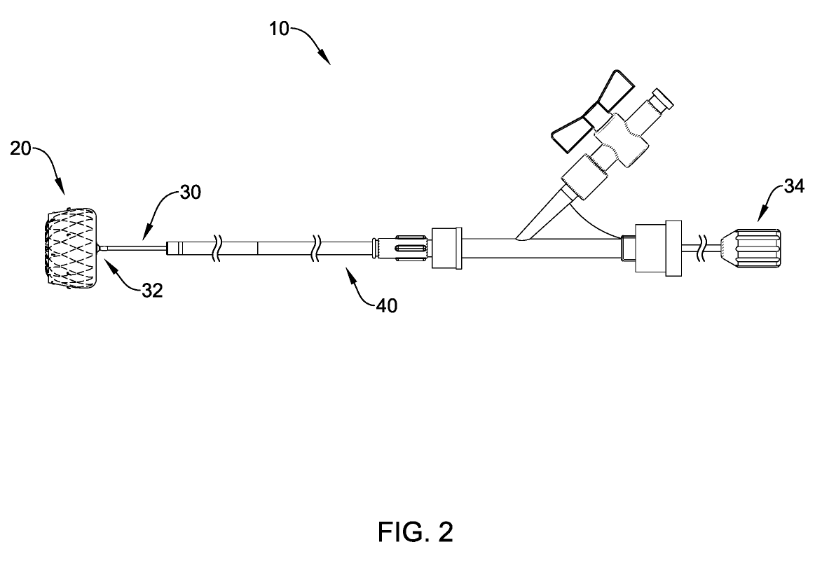

FIGS. 1-2 illustrate selected components and/or arrangements of an occlusive implant system 10 for occluding a left atrial appendage. It should be noted that in any given figure, some features of the occlusive implant system 10 may not be shown, or may be shown schematically, for simplicity. Additional details regarding some of the components of the occlusive implant system 10 may be illustrated in other figures in greater detail. The occlusive implant system 10 may be used to percutaneously deliver and/or deploy a variety of medical implants (e.g., a cardiovascular implant, an occlusive implant, a replacement heart valve implant, etc.) to one or more locations within the anatomy, including but not limited to, in some embodiments, the heart.

The occlusive implant system 10 may comprise a core wire 30. The occlusive implant system 10 may comprise an occlusive implant 20 releasably couplable to and/or disposable at a distal end 32 of the core wire 30. In at least some embodiments, the occlusive implant 20 may be configured to occlude the left atrial appendage. The left atrial appendage is attached to and in fluid communication with the left atrium of the patient’s heart. The left atrial appendage may have a complex geometry and/or irregular surface area. In some patients, the left atrial appendage may have a plurality of lobes and/or recesses extending in different directions.

In some embodiments, the occlusive implant system 10 may include a delivery sheath 40 having a lumen 42 (e.g., FIG. 1) extending from a proximal opening to a distal opening. In some embodiments, the core wire 30 may be slidably disposed within the lumen 42 of the delivery sheath 40. The core wire 30 may have a proximal end 34 disposed proximal of the delivery sheath 40. In some embodiments, the proximal end 34 of the core wire 30 may include a knob and/or a handle configured to manipulate and/or move the core wire 30 and/or the occlusive implant 20. In some embodiments, the proximal end 34 of the core wire 30 may include a knob and/or a handle configured to manipulate and/or move the core wire 30 and/or the occlusive implant 20 relative to the delivery sheath 40. In some embodiments, the delivery sheath 40 may be sized and configured to deliver the occlusive implant 20 to the left atrial appendage.

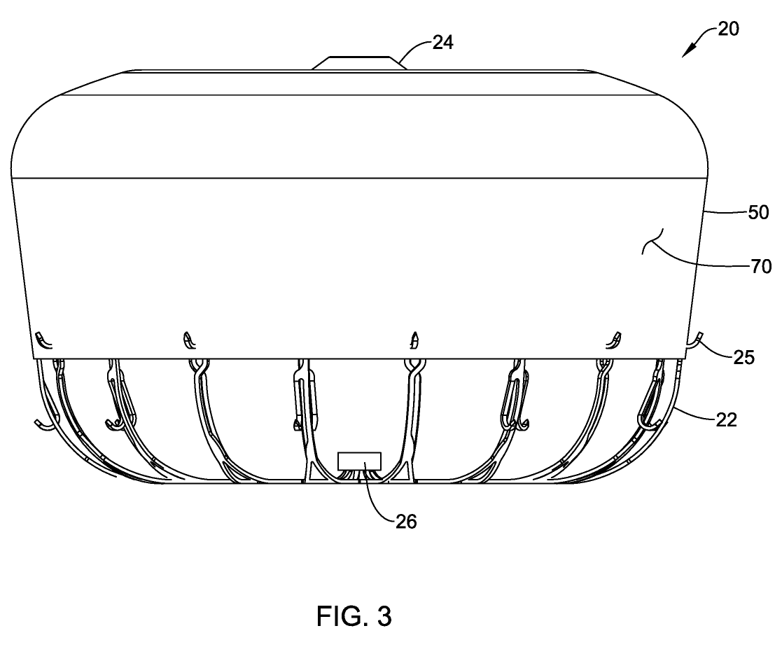

The occlusive implant 20 may include an expandable framework 22 (e.g., FIG. 3) configured to shift between a collapsed configuration (e.g., FIG. 1) and a deployed configuration (e.g., FIGS. 2-3). In some embodiments, the occlusive implant 20 may be disposed within the lumen 42 of the delivery sheath 40 proximate the distal opening in the collapsed configuration. In some embodiments, the delivery sheath 40 may constrain the occlusive implant 20 and/or the expandable framework 22 in the collapsed configuration. In some embodiments, the occlusive implant 20 and/or the expandable framework 22 may be configured to shift from the collapsed configuration to the deployed configuration when the occlusive implant 20 is disposed distal of the distal opening of the lumen 42 and/or the delivery sheath 40, and/or when the occlusive implant 20 is unconstrained. In some embodiments, the occlusive implant 20 and/or the expandable framework 22 may be configured to shift from the collapsed configuration to the deployed configuration when the occlusive implant 20 is unconstrained by the delivery sheath 40. In at least some embodiments, the expandable framework 22 may be self-biased toward the deployed configuration.

In some embodiments, the core wire 30 may be slidably and/or rotatably disposed within the lumen 42 of the delivery sheath 40. In some embodiments, the proximal end 34 of the core wire 30 may extend proximally of a proximal end of the delivery sheath 40 and/or the proximal opening of the lumen 42 for manual manipulation by a clinician or practitioner. In some embodiments, the occlusive implant 20 may be removably attachable, joinable, securable, or otherwise connectable to the distal end 32 of the core wire 30. The core wire 30 may be configured to and/or may be capable of axially translating the occlusive implant 20 relative to the delivery sheath 40. The delivery sheath 40 and/or the core wire 30 may have a selected level of axial stiffness and/or pushability characteristics while also having a selected level of flexibility to permit navigation through the patient’s vasculature.

Some suitable, but non-limiting, examples of materials for the occlusive implant system 10, the core wire 30, the delivery sheath 40, and/or the occlusive implant 20, etc. are discussed below.

Turning now to FIG. 3, the occlusive implant 20 may comprise an expandable framework 22 configured to shift along a longitudinal axis between the collapsed configuration and the deployed configuration. In the collapsed configuration, the expandable framework 22 may be axially elongated and/or radially compressed. In the deployed configuration, the expandable framework 22 may be axially shortened and/or radially expanded. The expandable framework 22 may comprise a plurality of interconnected struts defining a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements. In some embodiments, the plurality of interconnected struts may converge, join, and/or connect at intersections or nodes.

The plurality of interconnected struts may be formed and/or cut from a tubular member. In some embodiments, the plurality of interconnected struts may be integrally formed and/or cut from a unitary member. In some embodiments, the plurality of interconnected struts may be integrally formed and/or cut from a unitary tubular member and subsequently formed and/or heat set to a desired shape in the deployed configuration. In some embodiments, the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the plurality of interconnected struts include laser cutting, machining, punching, stamping, electro discharge machining (EDM), chemical dissolution, etc. Other means and/or methods are also contemplated.

In some embodiments, the expandable framework 22 may be compliant and substantially conform to and/or be in sealing engagement with the shape and/or geometry of a wall of the left atrial appendage in the deployed configuration. In some embodiments, the occlusive implant 20 may expand to a size, extent, or shape less than or different from a maximum unconstrained extent, as determined by the surrounding tissue and/or wall of the left atrial appendage. In some embodiments, reducing a thickness of various elements of the expandable framework 22 may increase the flexibility and compliance of the expandable framework 22 and/or the occlusive implant 20, thereby permitting the expandable framework 22 and/or the occlusive implant 20 to conform to the tissue around it, rather than forcing the tissue to conform to the expandable framework 22 and/or the occlusive implant 20. In some embodiments, the expandable framework 22 and/or the occlusive implant 20 may be stronger and/or less compliant, and thus the expandable framework 22 and/or the occlusive implant 20 may force the tissue of the left atrial appendage to conform to the expandable framework 22 and/or the occlusive implant 20 in the deployed configuration. Other configurations are also contemplated.

In some embodiments, the occlusive implant 20 and/or the expandable framework 22 may comprise a plurality of anchoring elements 25. In some embodiments, the plurality of anchoring elements 25 may extend radially outward from the expandable framework 22 in the deployed configuration. In at least some embodiments, the plurality of anchoring elements 25 may be configured to engage with tissue and/or may be configured to secure the occlusive implant 20 and/or the expandable framework 22 to tissue at a target site (e.g., the left atrial appendage, etc.). In some embodiments, the plurality of anchoring elements 25 may be configured to prevent dislodgement and/or ejection of the occlusive implant 20 from the target site and/or the left atrial appendage.

In some embodiments, the occlusive implant 20 and/or the expandable framework 22 may comprise a proximal hub 24 and/or a distal hub 26. The longitudinal axis of the expandable framework 22 may extend from the proximal hub 24 to the distal hub 26. In at least some embodiments, the proximal hub 24 and/or the distal hub 26 may be centered on and/or coaxial with the longitudinal axis. The plurality of interconnected struts may be joined together at and/or fixedly attached to the proximal hub 24 and/or the distal hub 26. In some embodiments, the proximal hub 24 and/or the distal hub 26 may be fixedly attached to the expandable framework 22 and/or the plurality of interconnected struts, such as by welding, adhesive bonding, brazing, soldering, etc. The proximal hub 24 may be configured to releasably connect, couple, and/or attach the occlusive implant 20 and/or the expandable framework 22 to the distal end 32 of the core wire 30 (e.g., FIGS. 1-2). In some embodiments, the proximal hub 24 may include internal threads configured to rotatably and/or threadably engage external threads formed on and/or at the distal end 32 of the core wire 30. Other configurations and/or structures for releasably securing the occlusive implant 20 to the core wire 30, including but not limited to pins, springs, detents, etc., are also contemplated.

In some embodiments, the occlusive implant 20 may comprise an occlusive fabric 50 secured to, attached to, connected to, disposed on, disposed over, disposed around, and/or disposed radially outward of a proximal portion of the expandable framework 22 and/or the plurality of interconnected struts. In some embodiments, the occlusive fabric 50 may be attached to the proximal hub 24 and/or may be attached to the expandable framework 22 at the proximal hub 24. In some embodiments, the occlusive fabric 50 may extend radially outward from and/or may extend distally from the proximal hub 24. In some embodiments, the occlusive fabric 50 may be attached and/or secured to the expandable framework 22 at a plurality of discrete locations. In some embodiments, one or more anchoring element(s) of the plurality of anchoring elements 25 may extend through the occlusive fabric 50. In some embodiments, the one or more anchoring element(s) of the plurality of anchoring elements 25 extending through the occlusive fabric 50 may attach and/or secure the occlusive fabric 50 to the expandable framework 22.

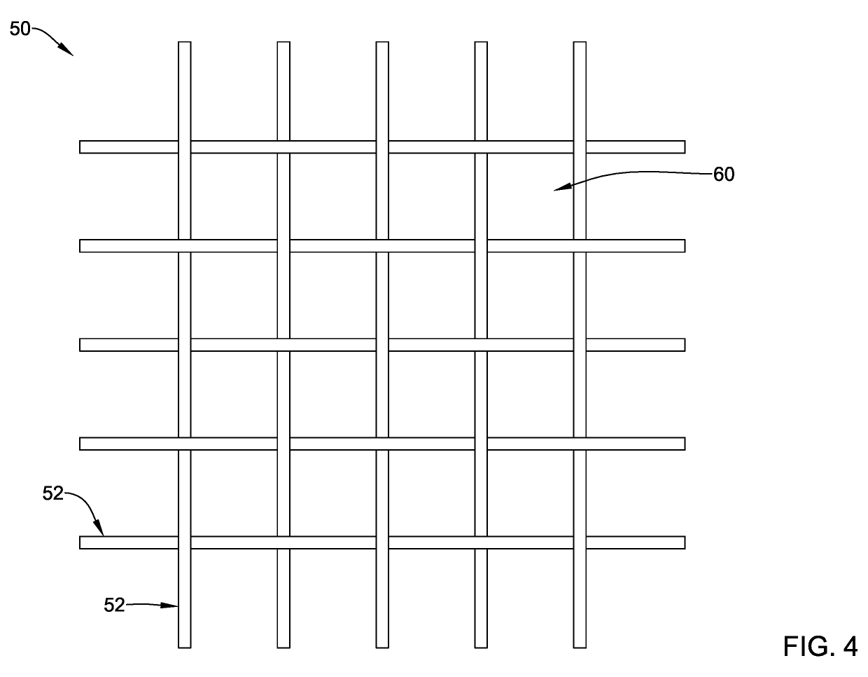

Selected aspects of the occlusive fabric 50 are shown in FIG. 4 in greater detail. In some embodiments, the occlusive fabric 50 may comprise and/or may be formed from a plurality of filaments 52. In some embodiments, the occlusive fabric 50 may be a woven structure, a knitted structure, a fabric structure, a textile structure, and/or a membrane or film having a plurality of apertures formed therein and/or extending therethrough. For the purpose of illustration only, the occlusive fabric 50 is shown in FIG. 4 as a woven structure. The occlusive fabric 50 is shown with a one-over, one-under weave pattern, but other weave patterns and/or configurations are also contemplated. Additionally, it shall be understood that in some embodiments, a knitted structure, which may include warp knitting, weft knitting, woven knitting, etc., may be preferred, even though such structure is not expressly illustrated. Various stitch patterns are also contemplated. In one non-limiting example, the occlusive fabric 50 may be formed with a single bar warp knit configuration having a 1-0/1-2 stitch pattern. Other configurations are also contemplated.

In some embodiments, the occlusive fabric 50 may comprise a plurality of pores 60 (e.g., a plurality of openings, and/or apertures) defined by the plurality of filaments 52. In some embodiments, the plurality of pores 60 (e.g., a plurality of openings, and/or apertures) may extend through the occlusive fabric 50 from a first side to a second side. In some alternative embodiments, the occlusive fabric 50 may be non-porous.

In some embodiments, the occlusive fabric 50 may be permeable or impermeable to blood and/or other fluids, such as water. In some embodiments, the occlusive fabric 50 may be designed, sized, and/or configured to prevent thrombus and/or embolic material from passing out of the left atrial appendage into the left atrium and/or the patient’s bloodstream. In some embodiments, the occlusive fabric 50 may promote endothelization after implantation, thereby effectively and/or permanently removing the target site (e.g., the left atrial appendage, etc.) from the patient’s circulatory system.

In some embodiments, the occlusive fabric 50 may include a surface treatment configured to promote endothelization on and/or across the occlusive fabric 50. In some embodiments, the occlusive fabric 50 may include the surface treatment disposed on and/or surrounding a portion of an outer surface and/or a proximally facing surface of the occlusive fabric 50. In some embodiments, the occlusive fabric 50 may include the surface treatment disposed on and/or surrounding an entire outer surface and/or an entire proximally facing surface of the occlusive fabric 50. In some embodiments, the occlusive fabric 50 may be elastic and/or stretchable to accommodate changes in shape and/or size of the expandable framework 22 when the expandable framework 22 is shifted toward and/or into the expanded configuration.

In some embodiments, the occlusive fabric 50 may comprise shape memory characteristics such that the occlusive fabric 50 may be configured to shift from a first configuration toward and/or to a second configuration when exposed to a predetermined stimulus. In some embodiments, the second configuration may define a permanent shape of the occlusive fabric 50. In some embodiments, the first configuration may define a temporary shape of the occlusive fabric 50. In some embodiments, the occlusive fabric 50 may be disposed in the first configuration (e.g., the temporary shape) during delivery and/or before implantation. In some embodiments, the occlusive fabric 50 may be configured to shift from the first configuration (e.g., the temporary shape) toward and/or to the second configuration (e.g., the permanent shape) after deployment and/or after implantation, wherein the occlusive fabric 50 is exposed to the predetermined stimulus during and/or after deployment and/or implantation. As such, in at least some embodiments, the first configuration may be a collapsed configuration commensurate with, compatible with, and/or complimentary to the collapsed configuration of the expandable framework 22.

In some embodiments, the predetermined stimulus may be a temperature greater than 37 degrees Celsius (C). Other ranges and/or values are also contemplated. In some embodiments, the predetermined stimulus may be a fluid, such as water, saline solution, blood, etc. Since the occlusive implant 20 and/or the occlusive fabric 50 is intended for intravascular use, the fluid shall be biocompatible. Other configurations and/or stimuli are also contemplated. In some embodiments, the predetermined stimulus may be a combination of two or more different stimuli (e.g., a fluid having a temperature greater than 37 degrees C, etc.). In some embodiments, the occlusive fabric 50 may be exposed to a temperature less than 37 degrees C, and/or dried, and/or otherwise affected (e.g., relatively cool temperature, dehydration, etc.) to set the occlusive fabric 50 in the first configuration and/or the temporary shape. Thereafter, upon exposure to the predetermined stimulus, the occlusive fabric 50 may be configured to shift toward and/or to the second configuration and/or the permanent shape.

In some embodiments, the occlusive fabric 50 may be self-biased toward a collapsed configuration in the second configuration (e.g., the permanent shape), as seen in FIG. 3. In some embodiments, such as that shown in FIG. 3 for example, the occlusive fabric 50 may be self-biased radially inward against the expandable framework 22. In some embodiments, the occlusive fabric 50 may be self-biased toward an expanded configuration in the first configuration (e.g., the temporary shape), and self-biased toward a collapsed configuration in the second configuration (e.g., the permanent shape). For example, the occlusive fabric 50 may be self-biased radially outward toward the extended configuration during implantation such that upon initial release from the delivery sheath 40, the occlusive fabric 50 is self-biased radially outward against a wall of the left atrial appendage. After being exposed to the predetermined stimulus, the occlusive fabric 50 may be self-biased radially inward against the expandable framework 22, as seen in FIG. 3.

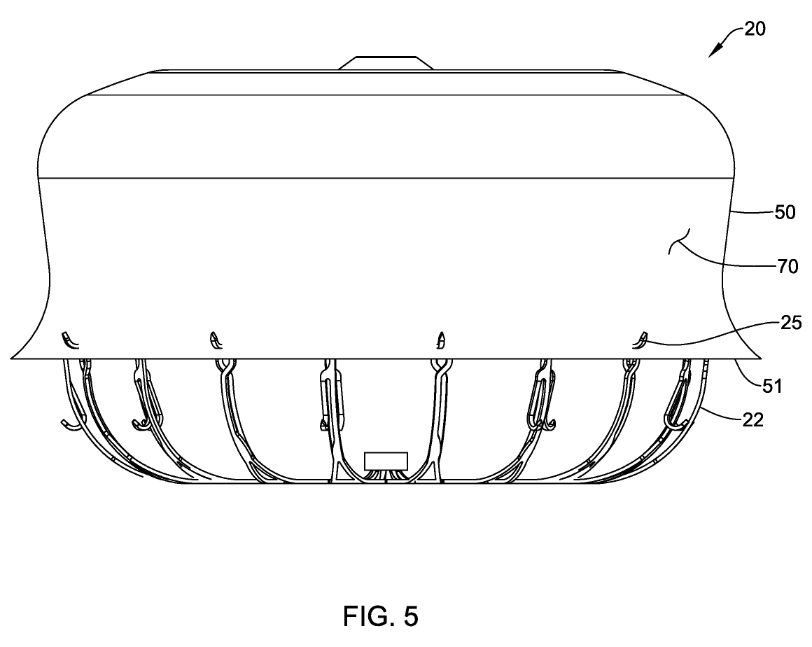

In some embodiments, the occlusive fabric 50 may be self-biased toward a collapsed configuration in the first configuration (e.g., the temporary shape), as seen in FIG. 1, and self-biased toward an extended configuration in the second configuration (e.g., the permanent shape), as seen in FIG. 5. For example, the second configuration (e.g., the permanent shape) of the occlusive fabric 50 may correspond to a flat sheet, such as an as-manufactured shape. In the configuration shown in FIG. 5, portions of the occlusive fabric 50 are constrained against the expandable framework 22 by the plurality of anchoring elements 25, thereby causing a distal end 51 or a free end of the occlusive fabric 50 to billow or scallop outward between adjacent anchoring elements of the plurality of anchoring elements 25 as the occlusive fabric 50 attempts to return to the second configuration (e.g., the permanent shape) of a flat sheet. The distal end 51 or the free end of the occlusive fabric 50 may be self-biased radially outward from the expandable framework 22 and/or against the wall of the left atrial appendage, thereby enhancing the seal between the occlusive implant 20 and/or the occlusive fabric 50 and the wall of the left atrial appendage.

Several different configurations are contemplated for providing the occlusive fabric 50 with shape memory characteristics. In some embodiments, the occlusive fabric 50 may optionally comprise a coating 70 formed from a shape memory polymer. The coating 70 may be disposed on an outer surface of the occlusive fabric 50, as shown in FIGS. 3 and 5 for example. In some alternative embodiments, the coating 70 may be disposed on an inner surface of the occlusive fabric 50. In some further alternative embodiments, the coating 70 may be disposed on both the inner surface and the outer surface of the occlusive fabric 50. In some embodiments, the coating 70 may be disposed on only a portion of the occlusive fabric 50 (e.g., a first portion of the occlusive fabric 50 may comprise the coating 70 and a second portion of the occlusive fabric 50 may be devoid of the coating 70). Other configurations are also contemplated.

In some embodiments, the occlusive fabric 50 may comprise and/or may be formed entirely from non-shape memory materials. For example, the plurality of filaments 52 may be formed from one or more non-shape memory materials. The coating 70 comprising a shape memory polymer may then be applied to the occlusive fabric 50 to impart shape memory characteristics, bias, and/or a shape memory effect to the occlusive fabric 50. In some embodiments, the shape memory characteristics, the bias, and/or the shape memory effect may be provided solely by the coating 70. Alternatively, in some embodiments, the occlusive fabric 50 may be formed from multiple different materials, including shape memory materials and non-shape memory materials, as discussed herein, such that the shape memory characteristics, the bias, and/or the shape memory effect may be provided by a combination of the plurality of filaments 52 and the coating 70.

In some embodiments, the coating 70 and/or the shape memory polymer forming the coating 70 may be configured to react to the predetermined stimulus. In some embodiments, the coating 70 may comprise a catalyst, a surfactant, etc. and/or combinations thereof, configured to react to the predetermined stimulus. In some embodiments, the catalyst, the surfactant, etc. and/or combinations thereof may be configured to initiate and/or accelerate a reaction of the coating 70 with the predetermined stimulus.

In some embodiments, when exposed to the predetermined stimulus, the coating 70 may be configured to shift from the first configuration (e.g., the temporary shape) toward and/or to the second configuration (e.g., the permanent shape), thereby imparting a bias and/or a shape memory effect to the occlusive fabric 50 itself (or the first portion of the occlusive fabric 50, as appropriate). In some embodiments, the coating 70 may be configured to bias the occlusive fabric 50 radially inward against the expandable framework 22 when exposed to the predetermined stimulus (e.g., similar to FIG. 3). In some embodiments, the coating 70 may be configured to bias the occlusive fabric 50 radially outward when exposed to the predetermined stimulus (e.g., similar to FIG. 5). Other configurations are also contemplated.

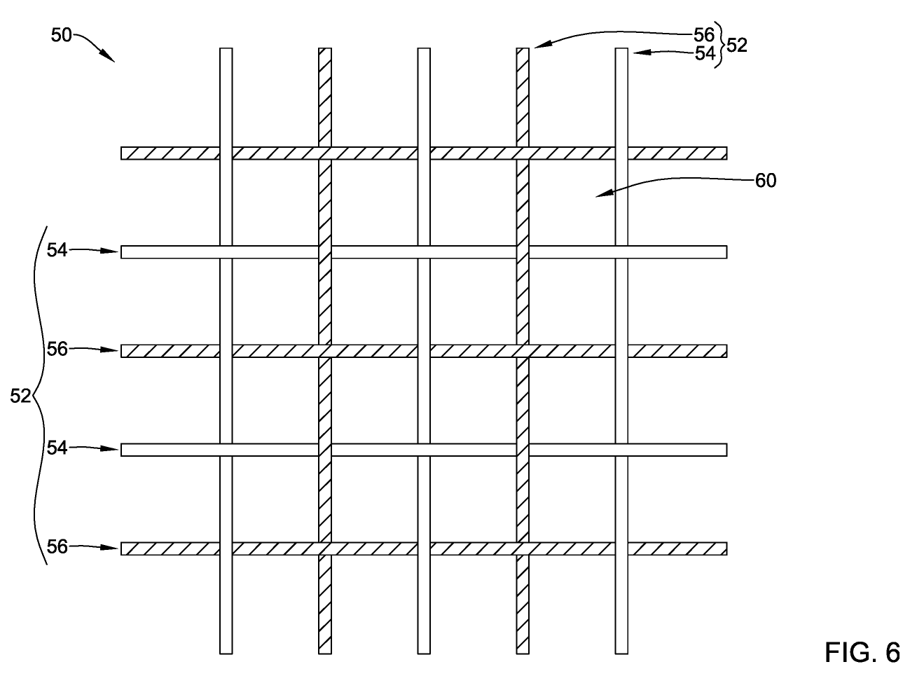

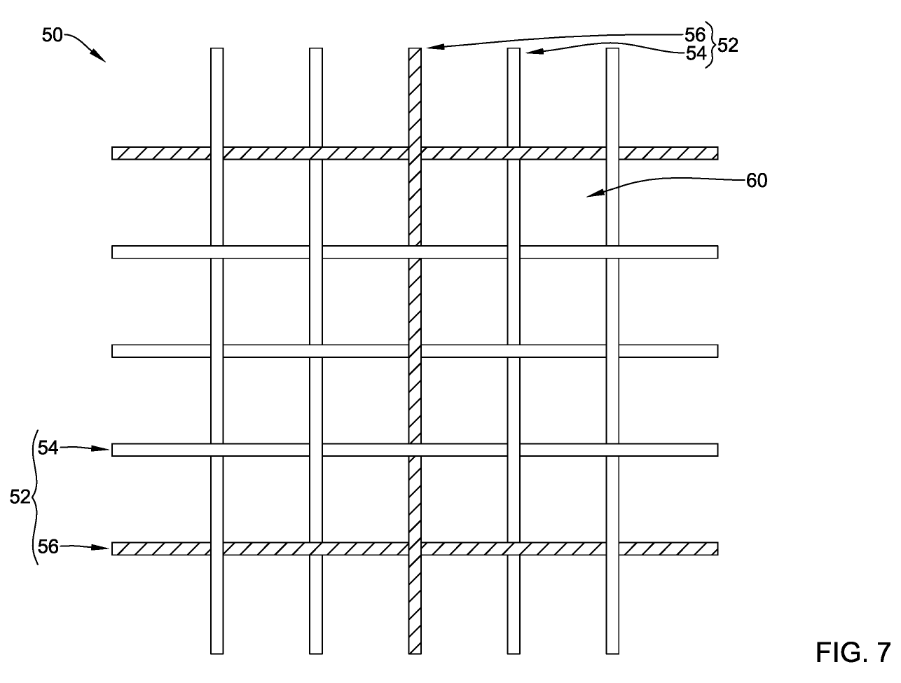

Turning now to FIGS. 6-7, in some embodiments, the plurality of filaments 52 may comprise a first subset of filaments 54 formed from a non-shape memory material and second subset of filaments 56 formed from a shape memory material. The second subset of filaments 56 may be configured to react to the predetermined stimulus to shift from the first configuration (e.g., the temporary configuration) toward and/or to the second configuration (e.g., the permanent configuration). The second subset of filaments 56 may be configured to exert a shape memory effect upon the occlusive fabric 50 when the occlusive fabric 50 and/or the second subset of filaments 56 is exposed to the predetermined stimulus.

In some embodiments, the second subset of filaments 56 and/or the shape memory material may comprise polyurethanes, polyesters such as (but not limited to) polycaprolactone (PCL), polylactic acid (PLA), poly-l-lactic acid (PLLA), poly lactic-co-glycolic acid (PLGA), polyglycolide (PGA), etc., and combinations or derivatives thereof, polyethers, and the like. Other configurations and/or materials having shape memory properties and/or characteristics are also contemplated. In some embodiments, it may be advantageous if the second subset of filaments 56 and/or the shape memory material has a glass transition temperature at and/or near normal human body temperature (e.g., about 37 degrees C). Other configurations are also contemplated.

In some embodiments, the second subset of filaments 56 may be evenly distributed within the occlusive fabric 50. In some embodiments, a density of the second subset of filaments 56 may vary within the occlusive fabric 50. In some embodiments, varying the density of the second subset of filaments 56 may vary and/or alter an amplitude of the shape memory effect exerted on the occlusive fabric 50 by the second subset of filaments 56. In some embodiments, an occlusive fabric 50 having a higher density of the second subset of filaments 56 may produce a greater amplitude of the shape memory effect than an occlusive fabric 50 having a relatively lower density of the second subset of filaments 56. For example, the amplitude of the shape memory effect may be greater in the configuration shown in FIG. 6 than in the configuration shown in FIG. 7. In this way, the density of the second subset of the filaments 56 and/or the amplitude of the shape memory effect may be “tuned” to produce a desired behavior and/or result of the shape memory effect within the occlusive fabric 50.

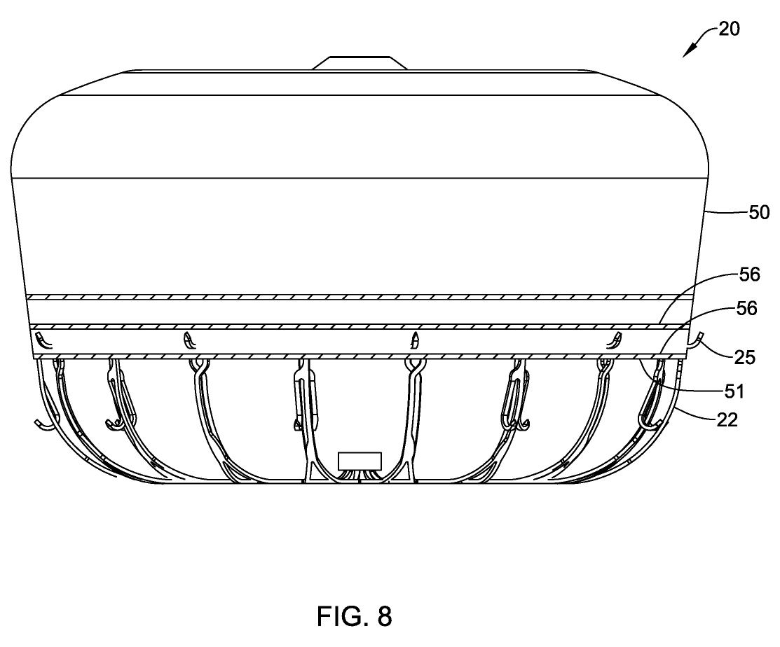

In some embodiments, the second subset of filaments 56 may be unevenly distributed within the occlusive fabric 50. In some embodiments, an uneven distribution of the second subset of filaments 56 may create a shape memory effect that is localized and/or concentrated within a particular portion of the occlusive fabric 50. For example, the second subset of filaments 56 may be concentrated along an edge of the occlusive fabric 50 (e.g., along the distal end 51 or the free end of the occlusive fabric 50), as seen in FIG. 8, to create a cinching effect configured to self-bias the distal end 51 or the free end of the occlusive fabric 50 against the expandable framework 22 when the occlusive fabric 50 and/or the second subset of filaments 56 is exposed to the predetermined stimulus.

In some embodiments, a method of making the occlusive fabric 50 may comprise intertwining the plurality of filaments 52 into and/or to form the occlusive fabric 50, wherein the occlusive fabric 50 is configured to shift from the first configuration (e.g., the temporary shape) toward and/or to the second configuration (e.g., the permanent shape) when exposed to the predetermined stimulus. In some embodiments, intertwining the plurality of filaments 52 may comprise weaving the plurality of filaments 52 together into and/or to form the occlusive fabric 50. In some embodiments, the plurality of filaments 52 may comprise knitting the plurality of filaments 52 together into and/or to form the occlusive fabric 50. Other configurations are also contemplated.

In some embodiments, the method of making the occlusive fabric 50 may comprise intertwining a first subset of filaments 54 and a second subset of filaments 56 to form the occlusive fabric 50. In some embodiments, the plurality of filaments 52 may comprise one or more additional subsets of filaments (e.g., a third subset of filaments, a fourth subset of filaments, etc.). In some embodiments, each subset of filaments of the plurality of filaments 52 may comprise and/or may be formed from a different material. Other configurations are also contemplated.

In some embodiments, the method of making the occlusive fabric 50 may comprise defining a permanent shape of the occlusive fabric 50 in the second configuration. In some embodiments, the permanent shape of the occlusive fabric 50 and/or the second configuration of the occlusive fabric 50 may be a flat sheet. In some embodiments, the permanent shape of the occlusive fabric 50 and/or the second configuration of the occlusive fabric 50 may be an as-manufactured shape.

In some embodiments, the method of making the occlusive fabric 50 may comprise setting the occlusive fabric 50 in the first configuration, wherein the first configuration defines the temporary shape of the occlusive fabric 50. In some embodiments, the first configuration (e.g., the temporary shape) of the occlusive fabric 50 may comprise a collapsed configuration and/or a delivery configuration. Other configurations are also contemplated. In some embodiments, setting the occlusive fabric 50 may comprise heat setting the occlusive fabric 50 in the first configuration. Other configurations are also contemplated.

In some embodiments, the method of making the occlusive fabric 50 may comprise coating the occlusive fabric 50 with a coating 70 comprising a shape memory polymer configured to react to the predetermined stimulus. In some embodiments, the method of making the occlusive fabric 50 may comprise coating the occlusive fabric 50 with the shape memory polymer configured to react to the predetermined stimulus. Other configurations are also contemplated.

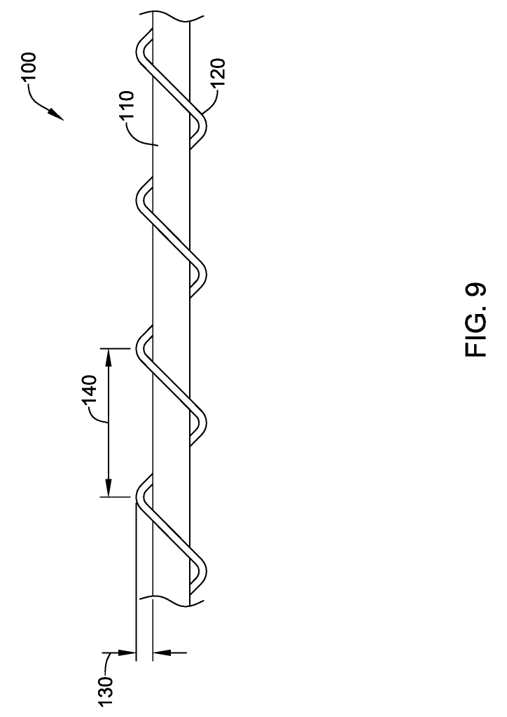

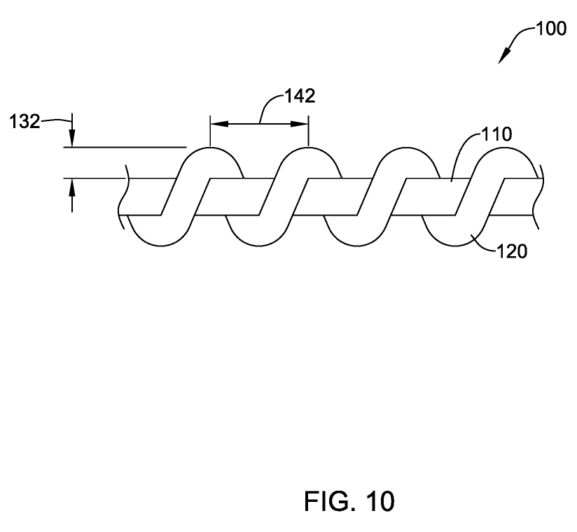

FIGS. 9-10 illustrate selected aspects of a shape memory yarn 100 and a method of making the shape memory yarn 100. In some embodiments, the occlusive fabric 50 (e.g., FIGS. 4, 6-7) may comprise and/or may be formed from the shape memory yarn 100. In some embodiments, the plurality of filaments 52 (e.g., FIGS. 4, 6-7) may comprise and/or may be formed from the shape memory yarn 100. In some embodiments, the second subset of filaments 56 (e.g., FIGS. 6-7) may be formed from the shape memory yarn 100. In some alternative embodiments, the first subset of filaments 54 may be formed from a first shape memory yarn and the second subset of filaments 56 may be formed from a second shape memory yarn different from the first shape memory yarn. Other configurations are also contemplated.

In some embodiments, the method of making the shape memory yarn 100 may comprise forming a core filament 110 from a first material comprising a polymer. In some embodiments, the core filament 110 and/or the first material may be formed from a composite material. In some embodiments, the core filament 110 and/or the first material may be formed from a non-polymer, such as a metallic material. In some embodiments, the core filament 110 and/or the first material may be formed from a non-shape memory material. Other configurations are also contemplated.

In some embodiments, the method of making the shape memory yarn 100 may comprise wrapping at least one shape memory filament 120 around the core filament 110. In some embodiments, the method of making the shape memory yarn 100 may comprise helically wrapping at least one shape memory filament 120 around the core filament 110. In at least some embodiments, the shape memory yarn 100 may comprise the core filament 110 formed from a first material comprising a shape memory polymer, and at least one shape memory filament 120 disposed helically around the core filament 110. In some embodiments, the at least one shape memory filament 120 may comprise a single shape memory filament. In some embodiments, the at least one shape memory filament 120 may comprise a plurality of shape memory filaments. The at least one shape memory filament 120 may be formed from a second material comprising a shape memory polymer.

In some embodiments, the at least one shape memory filament 120 may be configured to shift from a first configuration (e.g., a temporary shape) to a second configuration (e.g., a permanent shape) when exposed to a predetermined stimulus. In some embodiments, the predetermined stimulus may be a fluid (e.g., water, saline solution, blood, etc.). In some embodiments, the predetermined stimulus may be a temperature greater than 37 degrees Celsius (C). In some embodiments, the predetermined stimulus may be a fluid (e.g., water, saline solution, blood, etc.) or a temperature greater than 37 degrees Celsius (C). In some embodiments, the predetermined stimulus may be a combination of two or more different stimuli (e.g., a fluid having a temperature greater than 37 degrees C, etc.). Other configurations are also contemplated.

In some embodiments, the at least one shape memory filament 120 may define a first outer extent 130 from the core filament 110 in the first configuration (e.g., the temporary shape), as seen in FIG. 9, and a second outer extent 132 from the core filament 110 in the second configuration (e.g., the permanent shape), as seen in FIG. 10. In some embodiments, the at least one shape memory filament 120 may comprise a plurality of fiber branches extending radially outward therefrom, wherein the plurality of fiber branches may be compressed down against the at least one shape memory filament 120 in the first configuration and may be self-biased to extend outward from the at least one shape memory filament 120 in the second configuration. Other configurations are also contemplated.

In some embodiments, the at least one shape memory filament 120 may define a first spacing 140 between adjacent helical windings in the first configuration (e.g., the temporary shape), as seen in FIG. 9, and a second spacing 142 between adjacent helical windings in the second configuration (e.g., the permanent shape), as seen in FIG. 10. In some embodiments, the at least one shape memory filament 120 may be axially stretched and/or elongated in the first configuration. In some embodiments, the at least one shape memory filament 120 may be axially shortened in the second configuration. Other configurations are also contemplated.

Upon exposure to the predetermined stimulus, the at least one shape memory filament 120 may be configured to shift toward and/or to the second configuration. In some embodiments, upon exposure to the predetermined stimulus, the at least one shape memory filament 120 may be configured to enlarge, bulk up, plump up, etc. as the at least one shape memory filament 120 shifts toward and/or to the second configuration. In some embodiments, upon exposure to the predetermined stimulus, the at least one shape memory filament 120 may axial shorten as the at least one shape memory filament 120 shifts toward and/or to the second configuration. Other configurations are also contemplated.

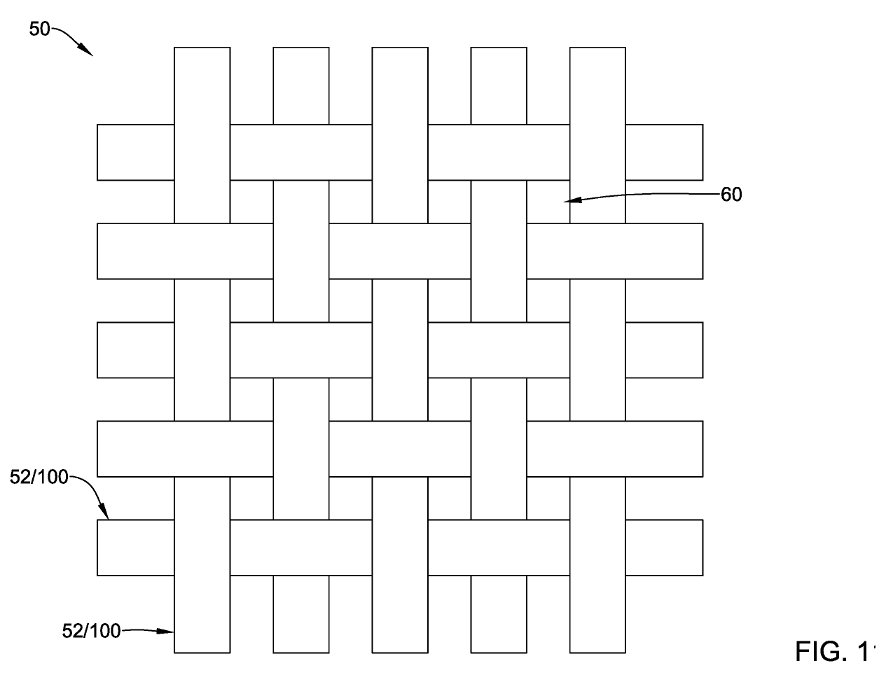

As discussed herein, in some embodiments, the occlusive fabric 50 may be formed from the shape memory yarn 100. In some embodiments, the occlusive fabric 50 may be formed from a plurality of shape memory yarns. Other configurations are also contemplated. When the occlusive fabric 50 is formed from the shape memory yarn 100 and/or the plurality of shape memory yarns, the occlusive fabric 50 in the first configuration may be formed similarly to the arrangement shown in FIG. 4 and the occlusive fabric 50 in the second configuration may be formed similarly to the arrangement shown in FIG. 11.

In some embodiments, each pore of the plurality of pores 60 (e.g., the plurality of openings, and/or apertures) extending through the occlusive fabric 50 may have a first size in the first configuration (e.g., FIG. 4) of between about 5 micrometers (microns) and about 500 micrometers, between about 50 micrometers and about 300 micrometers, between about 100 micrometers and about 220 micrometers, between about 140 micrometers and about 180 micrometers, and/or about 160 micrometers. In some embodiments, each pore the plurality of pores 60 (e.g., the plurality of openings, and/or apertures) extending through the occlusive fabric 50 may have a maximum size in the first configuration of about 1 millimeter (e.g., 1000 micrometers).

In some embodiments, each pore the plurality of pores 60 (e.g., the plurality of openings, and/or apertures) extending through the occlusive fabric 50 may have a maximum size in the second configuration of about 200 micrometers. In some embodiments, each pore the plurality of pores 60 (e.g., the plurality of openings, and/or apertures) extending through the occlusive fabric 50 may have a maximum size in the second configuration of about 100 micrometers. In some embodiments, each pore the plurality of pores 60 (e.g., the plurality of openings, and/or apertures) extending through the occlusive fabric 50 may have a maximum size in the second configuration of about 50 to 80 micrometers. Other configurations are also contemplated.

In some embodiments, each portion of the plurality of pores 60 (e.g., the plurality of openings, and/or apertures) may have a first cross-sectional area in the first configuration. In some embodiments, each pore of the plurality of pores 60 (e.g., the plurality of openings, and/or apertures) extending through the occlusive fabric 50 may have a second cross-sectional area less than the first cross-sectional area in the second configuration (e.g., FIG. 11). In some embodiments, as the occlusive fabric 50 shifts from the first configuration toward and/or to the second configuration, the shape memory yarn 100 may shift from the first configuration toward and/or to the second configuration.

In some embodiments, as the occlusive fabric 50 and/or the shape memory yarn 100 shifts from the first configuration toward and/or to the second configuration, the at least one shape memory filament 120 may shift from the first outer extent 130 (e.g., FIG. 9) toward and/or to the second outer extent 132 (e.g., FIG. 10), and/or the plurality of pores 60 may shrink and/or get smaller, as seen when comparing FIG. 11 to FIG. 4.

In some embodiments, the occlusive fabric 50, the coating 70, and/or the shape memory yarn 100 may comprise and/or provide drug-eluting characteristics. In some embodiments, the non-shape memory material, the shape memory material, the first material, and/or the second material described herein may comprise and/or may be selected for drug-eluting characteristics. In some embodiments, the non-shape memory material, the shape memory material, the first material, and/or the second material be configured to degrade in vivo to elute drugs and/or medicines. In some embodiments, the non-shape memory material, the shape memory material, the first material, and/or the second material be configured to degrade in response to an inflammatory response by the patient’s body to elute drugs and/or medicines. Other configurations are also contemplated.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the expandable framework, the occlusive fabric, the shape memory yarn, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. An occlusive implant, comprising:
a framework configured to shift between a collapsed configuration and an expanded configuration; and
an occlusive fabric secured to the framework;
wherein the occlusive fabric comprises shape memory characteristics such that the occlusive fabric is configured to shift from a first configuration to a second configuration when exposed to a predetermined stimulus.

2. The occlusive implant of claim 1, wherein the occlusive fabric is formed from a plurality of filaments comprising a first subset of filaments formed from a non-shape memory material and a second subset of filaments formed from a shape memory material.

3. The occlusive implant of claim 2, wherein the second subset of filaments is evenly distributed within the occlusive fabric.

4. The occlusive implant of claim 2, wherein the second subset of filaments is unevenly distributed within the occlusive fabric.

5. The occlusive implant of claim 4, wherein the second subset of filaments is concentrated along an edge of the occlusive fabric.

6. The occlusive implant of claim 1, wherein the occlusive fabric comprises a plurality of pores.

7. The occlusive implant of claim 6, wherein each pore of the plurality of pores has a first cross-sectional area in the first configuration and a second cross-sectional area less than the first cross-sectional area in the second configuration.

8. The occlusive implant of claim 1, wherein the occlusive fabric is self-biased toward a collapsed configuration in the first configuration and is self-biased toward an extended configuration in the second configuration.

9. The occlusive implant of claim 1, wherein the occlusive fabric is self-biased toward a collapsed configuration in the second configuration.

10. The occlusive implant of claim 1, wherein the occlusive fabric comprises a coating formed from a shape memory polymer configured to react to the predetermined stimulus.

11. The occlusive implant of claim 1, wherein the predetermined stimulus is a temperature greater than 37 degrees C.

12. The occlusive implant of claim 1, wherein the predetermined stimulus is a fluid.

13. A method of making an occlusive fabric having shape memory characteristics, comprising:
intertwining a plurality of filaments into an occlusive fabric, wherein the occlusive fabric comprises shape memory characteristics such that the occlusive fabric is configured to shift from a first configuration to a second configuration when exposed to a predetermined stimulus;
defining a permanent shape of the occlusive fabric in the second configuration; and
heat setting the occlusive fabric in the first configuration, wherein the first configuration defines a temporary shape of the occlusive fabric.

14. The method of claim 13, wherein the plurality of filaments comprises a first subset of filaments formed from a non-shape memory material and a second subset of filaments formed from a shape memory material, wherein the second subset of filaments is configured to react to the predetermined stimulus.

15. The method of claim 13, further comprising coating the occlusive fabric with a shape memory polymer configured to react to the predetermined stimulus.

16. The method of claim 13, wherein the predetermined stimulus is a fluid or a temperature greater than 37 degrees C.

17. A method of making a shape memory yarn, comprising:
forming a core filament from a first material comprising a polymer; and
wrapping at least one shape memory filament helically around the core filament, wherein the at least one shape memory filament is formed from a second material comprising a shape memory polymer;
wherein the at least one shape memory filament is configured to shift from a first configuration to a second configuration when exposed to a predetermined stimulus.

18. The method of claim 17, wherein the at least one shape memory filament defines a first outer extent from the core filament in the first configuration and a second outer extent from the core filament greater than the first outer extent in the second configuration.

19. The method of claim 17, wherein the at least one shape memory filament defines a first spacing between adjacent helical windings in the first configuration and a second spacing between adjacent helical windings less than the first spacing in the second configuration.

20. The method of claim 17, wherein the predetermined stimulus is a fluid or a temperature greater than 37 degrees C.
